(19) United States
(12) Patent Application Publication
(10) Pub. No.: US 2019/0314217 A1
(43) Pub. Date: Oct. 17, 2019

(54) INTIMATE SKIN CONDITIONER VEIL COMPRISING A NONWOVEN MATERIAL

(71) Applicant: Essity Hygiene and Health Aktiebolag, Göteborg (SE)

(72) Inventors: Magdalena Hörle, Göteborg (SE); Karin Alenius, Göteborg (SE); Shabira Abbas, Göteborg (SE); Chatrine Stridfeldt, Göteborg (SE)

(73) Assignee: Essity Hygiene and Health Aktiebolag, Göteborg (SE)

(21) Appl. No.: 16/313,577

(22) PCT Filed: Jun. 28, 2016
(86) PCT No.: PCT/SE2016/050655

§ 371 Date: Dec. 27, 2018

(51) Int. Cl. A61F 13/511; A61F 13/472; A61F 13/56; A61M 35/00; A61L 15/26; A61L 15/20; A61L 15/28; A61L 15/50

(52) U.S. Cl. CPC A61F 13/51113; A61F 13/472; A61F 13/5611; A61F 13/5116; A61F 2013/15943; A61L 15/26; A61L 15/20; A61L 15/28; A61L 15/50; A61M 35/10

(57) ABSTRACT

An intimate skin conditioner veil, configured to be positioned in an undergarment so that it covers the mons pubis in front of the anterior labia commissure of a female user, without covering any part of the vulval vestibule area of the user. The main portion may be reflection symmetric about a longitudinal central line, and the veil may include a garment facing layer arranged on a garment facing side of the veil, and a space creating layer arranged on a skin contacting side of the veil. The space creating layer comprises a nonwoven material arranged at least one the skin contacting side of the veil. The nonwoven material includes non-absorbent fibres. The fibres and/or the nonwoven material is/are coated with a lubricating coating composition configured for use in hygiene products at least on the skin contacting side of the veil to be in contact with skin of the user.

### TECHNICAL FIELD

The present disclosure relates to an intimate skin conditioner veil configured to be positioned in an undergarment so that it covers the V-zone of a female user, i.e. covering the mons pubis in front of the anterior labia commissure of a female user, without covering any part of the vulval vestibule area of the user.

### BACKGROUND ART

Shaving or removing hair from the genital area is an essential part of daily beauty care for today's women. However, removing hair from the genital area of a female user may leave the skin in that area swollen, dry and often irritated. Cremes and lotions can be used to relieve skin irritation, but may stain the undergarment of the user. The absence of hair in the genital area after hair removal may also lead to the garments of the user chafing the skin and to undesirable skin problems when the skin becomes moist due to the undergarment lying too closely against the skin.

Skin problems can also be caused by forces arising from physical/mechanical interaction between the undergarment or an absorbent product and the user's skin. Thus, for example chafing is caused due to extra friction between the undergarment or an absorbent product and skin of the user. There have been several studies to improve softness of absorbent products and especially nonwoven materials used on surfaces in contact with skin, such as topsheets of absorbent products. Today's absorbent products focus on providing products with a high degree of comfort by using nonwoven materials mainly providing a soft feeling towards the skin.

For example, WO 2008/147264 relates to a nonwoven material for use as a body facing sheet in an absorbent article. The nonwoven material comprises at least two layers. The layer directed against the user during use of the article comprises staple fibres has a coarseness less than 1.5 dtex. This part of the sheet feels soft to the user. It is described that a soft feeling may also be achieved using fibres/filaments such as cotton, viscose, or lyocell providing high softness and textile comfort. However, these fibres/filaments bind liquid, thus leaving a wet surface against the skin, which is not advantageous. US 2006/0121811 relates to an absorbent product wherein the surface in physical contact with the skin of the user comprises fine fibres to create a soft material against the skin. Moreover, WO 2007/114742 relates to a nonwoven topsheet material comprising microfibres in contact with the skin to improve softness against the skin.

### SUMMARY

The present disclosure aims at providing a product, which can avoid or mitigate the problems that may arise after hair removal in the V-zone, said V-zone being a part of the genital area of a female user comprised of mons pubis in front of the anterior labia commissure in the. The present disclosure thus relates to an intimate skin conditioner veil, which is configured to be positioned in an undergarment so that it covers the mons pubis in front of the anterior labia commissure of the female user, without covering any part of the vulval vestibule area of the user, wherein the veil has a longitudinal central line extending in the longitudinal direction of the veil, and a first extension in the longitudinal direction, between a front end and a rear end of the veil, and a second extension in a transverse direction perpendicular to the longitudinal central line, and wherein the veil comprises a front edge and first and second side edges. The veil comprises a main portion having a length L1 in the longitudinal direction of the veil. The veil has a total longitudinal length L2. The length L1 of the main portion is at least 50% of the total longitudinal length L2 of the veil, wherein the main portion is bounded by a straight front transversal line between front transition points and the first and second side edges, and tapers towards the rear end of the veil, the main portion preferably being reflection symmetric about the longitudinal central line. The veil has a garment facing side and a skin contacting side, the garment facing side and the skin contacting side facing away from one another. The veil further comprises a garment facing layer arranged on the garment facing side of the veil, and a space creating layer arranged on the skin contacting side of the veil.

The space creating layer arranged on a skin contacting side of the veil comprises a nonwoven layer comprising non-absorbent fibres having a coarseness of from 0.1 to 10 dtex, from 0.5 to 7 dtex or from 0.5 to 3 dtex. Furthermore, the fibres and/or the nonwoven material is/are coated with a lubricating coating composition configured for use in hygiene products at least on a surface arranged to be in contact with skin of a user during use of the intimate conditioner veil.

When using an intimate skin conditioner veil, friction occurs between the skin and the surface of the nonwoven material arranged to be in contact with skin of a user during use of the hygiene product. The friction between the nonwoven material and skin in presence of moisture/liquid is complex and even a very small amount of moisture has a negative impact on the measured friction. The amount of moisture may be so small that the nonwoven material is experienced as dry when touched. In this case moisture may be present only between the fibres and the skin, each fibre-skin interaction due to the moisture is referred to as a wet contact. The wet contacts are caused by the build-up of menisci between the skin and fibre of the nonwoven material in wet contact with the skin.

As mentioned above, one reason for mechanical discomfort relates to clinging, i.e. the forces acting between the intimate skin conditioner veil and the skin in the presence of small amounts of moisture such as perspiration. Understanding the relation between the clinging forces, causing discomfort, and the properties of the nonwoven materials used in the intimate skin conditioner veil, helps create a material which minimizes these forces and their negative impact on the skin. Clinging can be described as a perpendicular force acting between a solid material and a support surface in the presence of a small amount of moist. An example of clinging is a shower curtain which can easily stick to skin in presence of small amount of moisture.

To reduce the friction on areas where nonwoven material lies against the skin of a wearer, it is preferable that the nonwoven material be designed so that at least wet friction is reduced. Wet friction is experienced between a wet or moist product and skin. Wet friction can occur even at small concentrations of moist or liquid presence in the product or in the boundary between the nonwoven material and the skin. Dry friction is experienced between a dry product and skin. The measurement method for determining the wet and dry friction will be described more in detail below.

By coating the nonwoven material with a lubricating coating composition configured for use in hygiene products at least on a surface of the space creating layer arranged to be in contact with skin of a user during use of the intimate skin conditioner veil (i.e., the skin contacting side of the veil), with non-absorbent fibres in the nonwoven material having a coarseness of from 0.1 to 10 dtex, from 0.5 to 7 dtex or from 0.5 to 3 dtex, the intimate skin conditioner veil exhibits a reduction in the wet friction between the product and the skin of the user.

The reduction is achieved by the fact that the lubricating coating composition increases the contact angle, thereby reducing menisci and the wet friction between the skin and nonwoven material.

Wet contacts are contacts between the fibres of the nonwoven material and the skin, where moisture is present only at the contact points and not in the pores of the nonwoven material. A material of coarser fibres results in fewer contact points with skin than does a material made of finer fibres. The combination of reducing the number of wet contacts and applying lubricating coating of moisture contributes to reducing the wet friction between the skin of the user and the nonwoven material.

It is an object of the present disclosure to provide an intimate veil comprising a space creating layer arranged on a skin contacting side of the veil, and the space creating layer comprising a nonwoven material arranged on the skin contacting side of the space creating layer, wherein the nonwoven material during the use of the intimate veil reduces the risk for mechanical discomfort due to friction between the intimate skin conditioner veil and skin.

Fine fibres with low coarseness are useful to have closest to the skin to improve softness, but are not necessarily suitable to have closest to the skin of a user in the presence of small amount of moisture. Fine fibres with low coarseness are soft in dry conditions, but when moisture/liquid is present fine fibres also present disadvantages.

Friction occurring between a nonwoven material and the skin of the user is different in the presence of liquid/moisture compared to when no liquid/moisture is present. Even a very small amount of moisture present originating from perspiration, sweat or other body fluids has an impact on the friction forces occurred between the nonwoven material and the skin of the user. The nonwoven material characteristics must thus be carefully chosen, so that the nonwoven material is able to minimize mechanical discomfort during the overall use of the product.

An important reason for mechanical discomfort relates to “clinging”, i.e. forces acting between the product and the skin in the presence of moisture (perspiration, sweat). Thus, it is an object of the disclosure to provide an intimate conditioner veil with a nonwoven material arranged at least on a surface arranged to be in contact with skin of a user during use which minimizes these forces and their negative impact on the skin.

The lubricating coating composition may include silicone oil, of which one preferable example is polydimethylsiloxane.

The lubricating coating composition may also include pentandiol, such as for example 1,5 pentandiol.

The lubricating coating composition may also include allantoin (e.g., 5-ureidohydantoin or glyoxyldiureide).

The lubricating coating composition may also include a polysaccharide, such as for example beta glucan or pectin. Pectin is a polysaccharide having a mean molecular weight of 20 000-400 000 u. The polysaccharide is mainly composed of galacturonic acid units, which can be esterified to varying degrees. Pectin is naturally present in the cell walls of all plants and functions as a binding agent. A common source for extracting pectin is citrus peel.

Another suitable lubricating coating is SKINMIMICS®, which comprises ceteareth-25, glycerin, cetyl alcohol, behenic acid, cholesterol, ceramide NP, ceramide NS, ceramide, EOS, ceramide EOP, ceramide AP, caprooyl, phytosphingosine, caprooyl and sphingosine.

Furthermore, another suitable lubricating coating composition is SK-INFLUX® V, which comprises ceramide NP, ceramide AP, ceramide EOP, phytosphingosine, cholesterol, sodium lauroyl, lactylate, carbomer and xanthan Gum.

The lubrication composition may include a combination of the above-described lubricating coating compositions.

These lubricating compositions are non-toxic and provide very good lubricating properties.

The lubricating coating composition may be coated in an amount of 10 ppm to 10%-by weight, based on the total weight of the nonwoven material.

The coating may be applied by means of printing or kiss rolling, and can be applied to 20-100% of the total surface area of the skin contacting side of the veil.

The nonwoven material may be present on all parts of an absorbent and/or hygiene product that are in contact with skin, such as for example on the topsheet of an absorbent article or in case of a diaper on the waist region, hip region, standing gathers, leg openings and belt. The absorbent and/or hygiene product provides for low friction between the skin of the user and the product, both when the area is substantially dry but also when the area is moist due to perspiration and sweat or the presence of other bodily fluids.

The nonwoven material may comprise spunbond, air laid, wet laid, carded, electro spun or meltblown nonwoven or any combination thereof. The nonwoven material may be a laminate or a combination of several types of nonwoven materials. The nonwoven material may comprise, for example, spunbond and meltblown nonwoven material in a combination and form a layered product spunbond-meltblown-spunbond (SMS) or spunbond-meltblown-meltblown-spunbond (SMMS).

The nonwoven material may have a basis weight, for example, from 8 to 80 g/m², 8 to 40 g/m², 8 to 30 g/m² or 8 to 20 g/m². Thus, a nonwoven material with sufficient basis weight to resist forces created by the friction is provided.

The hygiene product may be an absorbent product with skin contact such as a diaper, incontinence protection garment, sanitary napkin or panty shield. The absorbent product may comprise a chassis having a front and rear panel and an absorbent body having a wetting zone for receiving urine and other bodily fluids. The nonwoven material of the present disclosure is especially suitable to be used at regions outside an initial wetting zone or landing zone of the absorbent products. This means that the nonwoven material may at least partially cover the absorbent structure, but is preferably located outside the initial wetting zone or landing zone, i.e. an area in the crotch portion in which urine initially lands.

The nonwoven material is comprised in at least one region outside the wetting zone in the absorbent body for receiving urine and other bodily fluids. The nonwoven material is alternatively comprised in the regions outside the absorbent body.

The absorbent product may comprise a waist region, hip region, standing gathers, leg openings and belt. The nonwoven material is at least comprised in at least one of the waist region, hip region, standing gathers, leg openings and belt. These areas may include moist, e.g. perspiration/sweat, and friction between the nonwoven material and the skin of the user occurs with increased risk for chafing. This risk can be decreased by the use of the nonwoven material in these regions.

The belt can be attached to the chassis or the belt can be separate from the chassis while being arranged to be attachable to the chassis. The nonwoven material may at least be comprised in the belt on a side of the belt being arranged to be in contact with skin. Thus risk for chafing and skin problems can be reduced in the belt region.

An absorbent product may comprise a topsheet, an absorbent body and a backsheet, and wherein the nonwoven material is comprised in the topsheet and/or in the backsheet (for example around the leg openings in the backsheet being in contact with the skin) of the absorbent product. In this way risk for skin problems can be reduced in a large area of the absorbent product being in contact with the skin.

The nonwoven material as described above preferably can render lower friction values in presence of moisture, compared to a nonwoven material comprising fibres and/or nonwoven material without a coating with a lubricating coating composition and/or fibres having a finer coarseness than 0.1 dtex on a surface arranged to be in contact with skin of a user during use of the hygiene product. These friction values are measured according to a repeated stick and slip method which will be described in more detail below. A curve with friction value measurements is obtained in repeated runs using the method. The curve comprises a first slope having a positive coefficient illustrating increase in the friction values, a plateau, and a second slope having a negative coefficient illustrating decrease in the friction values. At the plateau, the friction values are substantially constant over the extension of the plateau. Small variations at the plateau as well as along the slopes are possible between individual values, but with a positive coefficient is meant that all individual values in the first slope together creates a positive coefficient, as well as all individual values in the second slope together creates a negative coefficient, as well as all individual values in the plateau together creates a plateau.

The present disclosure further relates to a hygiene product such as an intimate conditioner veil comprising a nonwoven material arranged to be in contact with skin of a user during use of the product. A nonwoven material has lower maximum friction value in presence of moisture, measured along a curve obtained in repeated runs with measurements according to a repeated stick and slip method, compared to a nonwoven material comprising fibres and/or nonwoven material without a coating with a lubricating coating composition and/or fibres having a finer coarseness than 0.1 dtex on a surface arranged to be in contact with skin of a user during use of the hygiene product. The friction values are obtained in repeated runs, and the obtained friction values during the run form a curve comprising a first slope having a positive coefficient illustrating increase in the friction values, a plateau, and a second slope having a negative coefficient illustrating decrease in the friction values. The obtained friction values during the run form a curve comprising a first slope having a positive coefficient illustrating increase in the friction values, a plateau illustrating essentially unchanged friction, and a second slope having a negative coefficient illustrating decrease in the friction values. Lower friction values render the hygiene product more skin friendly, and skin problems arising with the use of the hygiene product such as an intimate conditioner veil can be reduced.

The present disclosure further relates to the use of a nonwoven material in a hygiene product to reduce wet friction between the nonwoven material and skin of a user. The nonwoven material comprises non-absorbent fibres, wherein the fibres have a coarseness of from 0.1 to 10 dtex, from 0.5 to 7 dtex or from 0.5 to 3 dtex. The fibres and/or the nonwoven material is coated with a lubricating coating composition configured for use in hygiene products, for example an intimate conditioner veil, at least on a surface arranged to be in contact with skin of a user during use of the product. It has been surprisingly noted that the nonwoven material of this type with lower friction values render the product more skin friendly and skin problems arising with the use of the hygiene product can be reduced. The wet friction is measured between the surface of the nonwoven material and skin of a user of the product by the repeated stick and slip method described in the description. The nonwoven material may be used in an absorbent product chosen from a diaper, incontinence protection garment, sanitary napkin or panty shield. The nonwoven material is used in substantially non-absorbent regions of the absorbent product.

The intimate skin conditioner veil may preferably further comprise a rear edge which extends substantially in the transverse direction of the veil, at the rear end of the veil, whereby the first and second side edges extend between the front transition points and rear transition points, and the main portion extends in the longitudinal direction between the front transversal line and a straight rear transversal line between the rear transition points, wherein the straight rear transversal line is perpendicular to the longitudinal central line. The main portion may preferably have a front transversal width W1 and a rear transversal width W2, wherein the ratio W1/W2 is 1.1-6.6, preferably 1.5-4.2, more preferably 2.8-3.5. The ratio of the longitudinal lengths L1/L2 is preferably 0.5-1, more preferably 0.6-0.9, most preferably 0.75-0.85. The longitudinal length L1 of the main portion is preferably 60-170 mm, and the total longitudinal length L2 of the veil is preferably 60-220 mm, more preferably 80-150 mm, most preferably 90-120 mm. The main portion preferably has a front transversal width W1 of 100-240 mm, more preferably 140-210 mm, most preferably 155-175 mm and a rear transversal width W2 of 25-95 mm, more preferably 40-80 mm, most preferably 55-70 mm.

The main portion advantageously is tapered such that a straight line between the front transition point and the rear transition point, is inclined in relation to the rear transversal line at an angle v¹, which is greater than 90°, preferably 95-160°, more preferably 100-130°, most preferably 105-125°, and that an angle v² between said straight line and the front transversal line is v²=180°−v¹.

The first and second side edges may advantageously have a curved concave shape between the front and rear transition points. In addition, the main portion may comprise a front main portion located between the front transversal line and a straight intermediate transversal line, and a rear main portion located between the rear transversal line and the intermediate transversal line, wherein longitudinal length of the front main portion is the same as the longitudinal length of the rear main portion, and wherein the curved concave shape of the first and second side edges changes curvature at an intermediate transition point, but runs in the same direction of curvature, wherein the intermediate transition point is located in the rear main portion between the intermediate transversal line and the rear transversal line. The main portion preferably has an intermediate transversal width of 65-90 mm at the intermediate transversal line. Preferably, the first and second side edges changes curvature such that a straight line between is inclined in relation to the rear transversal line at an angle α, which is >90°, and a straight line between the front transition point and the intermediate transition point is inclined in relation to the front transversal line at angle β, which is <180°−α. The angle α is preferably smaller than the angle v¹ between the rear transversal line and the straight line between the rear transition point and the front transition point, and the angle β is preferably smaller than the angle v² between the straight line and the front transversal line.

The intimate skin conditioner veil should preferably have a Gurley stiffness of 5-300 mgf, more preferably 5-100 mgf. The space creating layer may preferably be a combined layer, comprising a loft layer and a top sheet, the loft layer being positioned between the garment facing layer and the top sheet. The loft layer of the space creating layer may suitably have a basis weight of 15-100 gsm and a thickness of 0.5-3.5 mm. Alternatively, the space creating layer may be a multilayer layer comprising a loft portion and a top sheet portion, the loft portion being positioned between the garment facing layer and the top sheet portion. The top sheet is suitably a nonwoven material comprising natural and/or synthetic fibres, and preferably has a basis weight of 8-100 gsm. The garment facing layer is suitably a breathable back sheet, preferably comprising a breathable plastic film or a nonwoven material, such as combinations of spunbond-meltblown layers. The intimate skin conditioner veil should preferably have a moisture vapour transmission rate greater than 1000 g/m²/24 h, preferably 1300-1600 g/m²/24 h. The thickness of the intimate skin conditioner veil is preferably 0.6-3.8 mm.

Fastening means may suitably be positioned on the garment facing side of the veil, preferably in the form of one or more adhesive or friction areas. The loft layer may be smaller than the garment facing layer and the top sheet. The layers comprised in the veil may suitably be connected by adhesive or ultrasound welded joints.

The intimate skin conditioner veil may further comprising a head portion extending between the front straight line and the front edge, which head portion may comprise at least one protuberance, suitably having a convex shape. The head portion may comprise at least one recess, suitably having a concave shape, and suitably being positioned symmetrically about the longitudinal central line. At least one protuberance may be located on each side of the recess. The intimate skin conditioner veil may also comprise a tail portion extending between the rear transversal line and the rear edge. The tail portion may comprise at least one protuberance and/or recess.

The intimate skin conditioner veil may advantageously comprise an additive composition in the form of a skin well-being agent or a carrier substance carrying a skin well-being agent. The additive composition is preferably comprised within or on the space creating layer.

### BRIEF DESCRIPTION OF THE DRAWINGS

Other features and advantages disclosed herein will become more apparent from the following detailed description of illustrative embodiments when read in conjunction with the attached drawings, wherein:

FIG. 1 shows an illustrative embodiment of a veil placed in a panty;

FIG. 2 shows a cross section of the female anatomy of a wearer and an illustrative embodiment of a veil covering the mons pubis in front of the anterior labia commissure of the wearer;

FIG. 3 is a schematic top view of an illustrative embodiment of an intimate skin conditioner veil;

FIG. 4 schematically illustrates the outer contour of another illustrative embodiment of an intimate skin conditioner veil;

FIG. 5 is a schematic top view of an illustrative embodiment of another intimate skin conditioner veil;

FIG. 6 shows an enlarged portion of the intimate skin conditioner veil shown in FIG. 5;

FIG. 7 is a schematic cross sectional view of an illustrative embodiment of an intimate skin conditioner veil;

FIG. 8 is a schematic cross sectional view of a multilayer space creating layer used in an illustrative embodiment of an intimate skin conditioner veil;

FIG. 9 schematically illustrates an illustrative embodiment of an intimate skin conditioner veil in which the loft layer is smaller than the other layers; and

FIG. 10 schematically shows an enlargement of an illustrative embodiment of a fibre in a nonwoven material in the presence of moisture;

FIG. 11 schematically shows friction measurements made with an illustrative embodiment of a nonwoven material with and without lubricating coating composition on fibres in the nonwoven material using the repeated stick and slip method;

FIG. 12 shows principle sketches of different force contributions associated with illustrative embodiments of the nonwoven material;

FIG. 13a schematically shows an illustrative embodiment of an instrument set up for a stick and slip measurement method; and

FIG. 13b shows measurement data obtained by the repeated stick and slip method for a number of illustrative embodiments of nonwoven materials comprising fibres with lubricating coating composition and without lubricating coating composition.

### DETAILED DESCRIPTION

The present disclosure relates to a product in the form of an intimate skin conditioner veil, which is configured to be positioned in an undergarment so that it covers the mons pubis in front of the anterior labia commissure of a female user, without covering any part of the vulval vestibule area of the user. The part of the female genital area comprised of the mons pubis in front of the anterior labia commissure of a female user, is referred to as the “V-zone”.

In an illustrative embodiment, the intimate skin conditioner veil is configured to be positioned in an undergarment so that it contacts the mons pubis in front of the anterior labia commissure of a female user, without contacting any part of the vulval vestibule area of the user.

Accordingly, the intimate skin conditioner veil is essentially different from common hygiene products, which are intended to protect the garments of the wearer, typically by absorbing various body fluids excreted by the wearer. One purpose of the present intimate skin conditioner veil is to protect the skin in the genital area while reducing the risk of absorption of vaginal fluids besides body perspiration. The veil is therefore substantially non-absorbent. By substantially non-absorbent it is meant that the veil can absorb 0.9% by weight sodium chloride solution in an amount of 0-2 times its own weight. For example, to be substantially non-absorbent the veil can be formed from any single material or combination of materials, which include but are not limited to materials composed of a nonwoven material, e g spunbonded, meltblown, carded, hydroentangled, wetlaid, materials essentially consisting of non-absorbent fibers, i.e. at least 95% of the fibers are non-absorbent fibers, such as at least 99%, or at least 100% of the fibers in the material are non-absorbent fibers. Non-absorbent fibers in the fibrous can be selected from polyolefins, polyesters, polyamides and blends and combinations thereof.

The hair in the genital area acts as a shield against external mechanical stress, and creates an air gap between the skin and the undergarment that contributes to some airiness. When most or all of the hair has been removed, the undergarment material tends to lie more closely against the skin and there is a greater risk that the area becomes moist, which may increase the risk of bacterial growth and odour problem. Skin that has been shaved can also be negatively affected by the shaving itself due to mechanical irritation of the hair follicles, which may lead to an increased risk of infections in the hair follicles. Skin irritation can also be caused by mechanical stress on the skin when the user is moving, as the garment material lies very close to the skin.

The intimate skin conditioner veil serves to act as a protective barrier between the skin and the undergarment material, and to create a space between the skin and the undergarment material. The veil has a garment facing side and a skin contacting side, the garment facing side and the skin contacting side facing away from one another. The veil comprises a garment facing layer arranged on the garment facing side of the veil, and a space creating layer arranged on the skin contacting side of the veil. The veil is configured to be positioned in the undergarment, and therefore preferably comprises fastening means positioned on the garment facing side, most preferably in the form of one or more adhesive or friction areas. For example, adhesive or friction areas can be formed from any single material or combination of materials, which include but are not limited to hooks, friction adhesives, clips, pressure sensitive fastening adhesive. The veil is preferably not attached to the skin of the wearer, and it is therefore preferably essentially free from any adhesive on the skin contacting side.

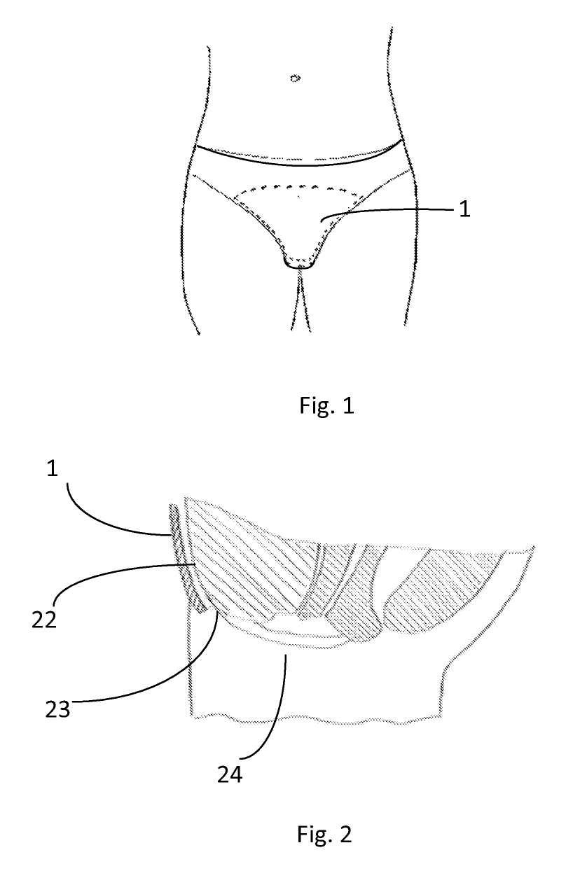

As indicated above, the veil is configured to be worn so that it covers the mons pubis in front of the anterior labia commissure of a female user, without covering any part of the vulval vestibule area of the user. The mons pubis, also known as mons Venus or mons veneris, is a rounded mass of fatty tissue found over the pubic symphysis of the pubic bones. In human females, the mons pubis forms the anterior portion of the vulva. It divides into the labia majora, on either side of the furrow known as the pudendal cleft that surrounds the labia minora, clitoris, urethra, vaginal opening, and other structures of the vulval vestibule. FIGS. 1 and 2 show how the veil is to be worn. FIG. 1 illustrates how the veil is placed in a panty, such that it covers the mons pubis in front of the anterior labia commissure. FIG. 2 shows a cross section of the female anatomy of a wearer and illustrates how the veil 1 covers the mons pubis 22 in front of the anterior labia commissure 23, and how it does not cover any part of the vulval vestibule area 24.

The intimate skin conditioner veil is configured to have a shape that essentially follows the anatomy of the wearer in the genital area. This is accomplished by a main portion of the veil. The main portion preferably has the general shape of a truncated triangle, so that the veil is wider at a front end towards the abdomen of the user, and narrower at a rear end towards the vulval vestibule area. Although a truncated triangular main portion is preferred, the veil may alternatively have a non-truncated triangular shape, with the base of the triangle at the front end and the top of the triangle at the rear end. Further, the intimate skin conditioner veil is preferably very pliable and very thin, as will be described in further detail below.

### Outer Contour

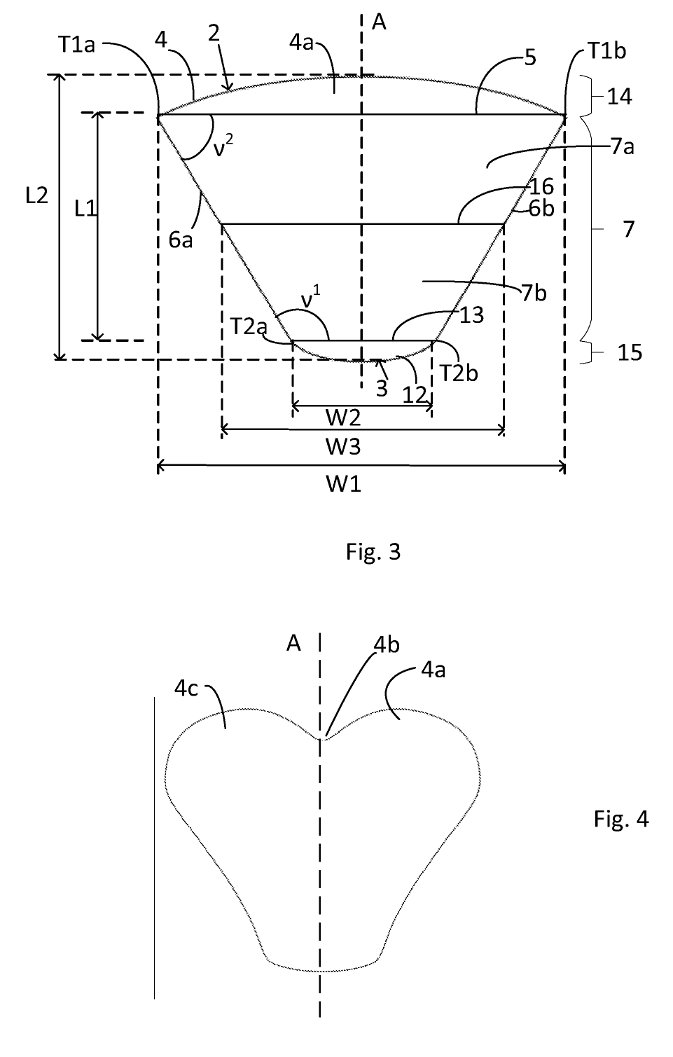

FIG. 3 illustrates an example of an intimate skin conditioner veil of the present disclosure. The veil has a longitudinal central line A extending in the longitudinal direction of the veil; a first extension in the longitudinal direction between a front end 2 and a rear end 3 of the veil; and a second extension in a transverse direction perpendicular to the longitudinal central line A. It comprises a front edge 4 and first and second side edges 6a, 6b, the front edge 4 intersecting the first side edge 6a at a first front transition point T1a and intersecting the second side edge 6b at a second front transition point T1b. The main portion 7 of the veil has a length L1 in the longitudinal direction of the veil, which is at least 50% of the total longitudinal length L2 of the veil 1. The main portion 7 is bounded by a straight front transversal line 5 between the first and second front transition points T1a, T1b, and by the first and second side edges 6a, 6b. The main portion 7 tapers towards the rear end 3 of the veil. The main portion 7 is preferably reflection symmetric about the longitudinal central line A. As indicated above, the main portion 7 is wider at its front end than at its rear end, and it preferably has a front transversal width W1 and a rear transversal width W2 chosen such that the ratio W1/W2 is 1.1-6.6, preferably 1.5-4.2, and more preferably 2.8-3.5.

The intimate skin conditioner veil 1 may further comprise a rear edge 12 which extends substantially in the transverse direction of the veil 1, at the rear end 3 of the veil, the rear edge 12 intersecting the first side edge 6a at a first rear transition point T2a and intersecting the second side edge 6b at a second rear transition point T2b. The first and second side edges 6a, 6b extend between respective ones of the first and second front transition points T1a, T1b and respective ones of the rear transition points T2a, T2b, and the main portion 7 extends in the longitudinal direction between the front transversal line 5 and a straight rear transversal line 13 between the first and second rear transition points T2a, T2b, and the straight rear transversal line 13 is perpendicular to the longitudinal central line A.

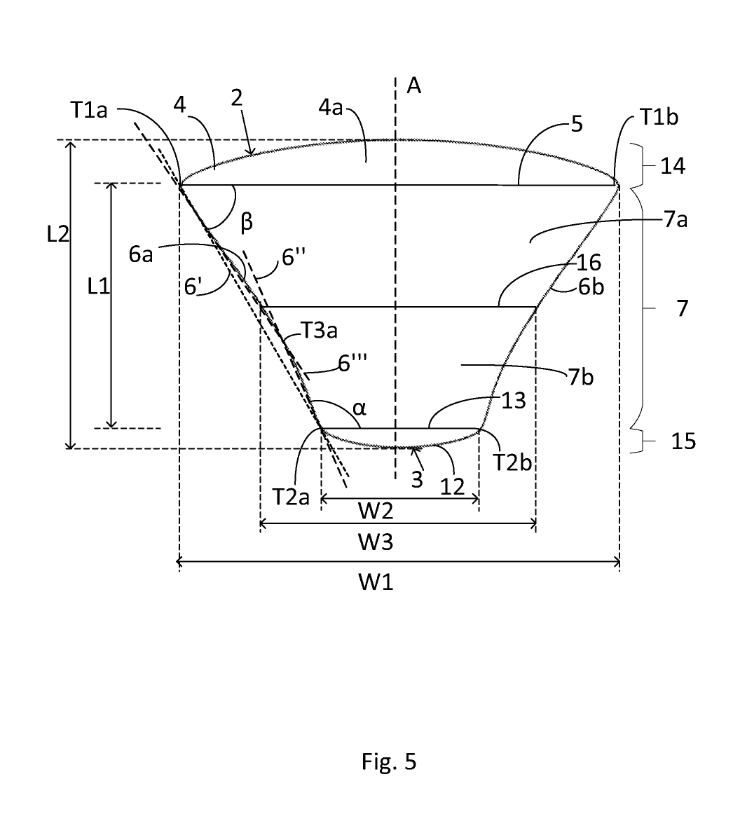

The first and second front transition points T1a, T1b and the first and second rear transition points T2a, T2b are located where the outer contour of the veil dramatically changes direction, between a generally longitudinal direction and a generally transversal direction. The front edge 4 of the veil meets the side edges 6a, 6b at the first and second front transition points T1a, T1b, respectively, and the rear edge 12 meets the side edges 6a, 6b at the first and second rear transition points T2a, T2b, respectively, such that the transition points form corners between the front and rear edges 4, 12 and the side edges 6a, 6b. These corners at the transition points can have a sharp shape or may be rounded. The veil shown in FIG. 3 has sharp corners at the front transition points, and the veil shown in FIG. 5 has rounded corners at the front transition points.

The intimate skin conditioner veil 1 may include a head portion 14 at the front end of the veil 1 and/or a tail portion 15. The head portion 14 extends between the front straight line 5 and the front edge 4 of the veil 1. The front edge 4 of the veil 1 is then also the front edge of the head portion 14. The head portion 14 can have various shapes, and can comprise at least one protuberance 4a, which may have a convex shape, as shown in FIG. 3. The head portion 14 may also comprise at least one recess 4b, which may have a concave shape, and which may be positioned symmetrically about the longitudinal central line A, as shown in FIG. 4. The head portion 14 may include at least one protuberance 4a, 4c located on each side of the recess 4b. FIG. 4 illustrates an example of a veil, in which one protuberance 4a, 4c is located on each side of a centrally positioned recess 4b, giving the veil 1 an outer contour resembling a rounded heart. Alternatively, a larger number of protuberances and recesses can be comprised in the head portion 14, for example, but not limited to 1-10 protuberances, and 1-9 recesses. The tail portion 15 extends between the rear transversal line 13 and the rear edge 12. The tail portion 15 portion can have various shapes, and may for example comprise at least one protuberance 12a and/or recess. The head and tail portions 14, 15 serve to further adapt the veil 1 to the anatomy of the wearer, by providing curved front and rear end contours. Fewer protuberances and recesses are preferred for manufacturing reasons, since a veil having a low number of protuberances and recesses in head or veil portion, may lead to a less complicated manufacturing process, while more protuberances and recesses provide a softer edge of the veil, such that the contour of the veil can follow the body shape of the user in an improved manner.

When the veil 1 includes a head portion 14 and/or a tail portion 15, the total longitudinal length L2 of the veil is the combined longitudinal length L1 of the main portion and the longitudinal lengths of the head portion and/or the tail portion. The ratio L1/L2 is preferably 0.5-1, more preferably 0.6-0.9, most preferably 0.75-0.85 so as to follow the anatomy of the wearer. The longitudinal length L1 of the main portion 7 is preferably 60-170 mm, and the total longitudinal length L2 of the veil is 60-220 mm, more preferably 80-150 mm, most preferably 90-120 mm so as to follow the anatomy of the wearer. The main portion preferably has a front transversal width W1 of 100-240 mm, more preferably 140-210 mm, most preferably 155-175 mm and a rear transversal width W2 of 25-95 mm, more preferably 40-80 mm, most preferably 55-70 mm so as to follow the anatomy of the wearer. By choosing the length and width of the veil according to the anatomy of the wearer, it can be ensured that the main portion is sufficiently large to cover the mons pubis in front of the anterior labia commissure.

In order to follow the anatomy of the wearer more closely, the main portion 7 may be tapered such that a straight line 6′ between the first front transition point T1a and the first rear transition point T2a is inclined in relation to the front transversal line 5 at an angle v², which is smaller than 90°, preferably 20-85°, more preferably 50-80°, most preferably 55-75°. The line 6′ is inclined in relation to the rear transversal line 13 at an angle v¹, which is greater than 90°, preferably 95-160°, more preferably 100-130°, most preferably 105-125°, such that v²=180°−v¹.

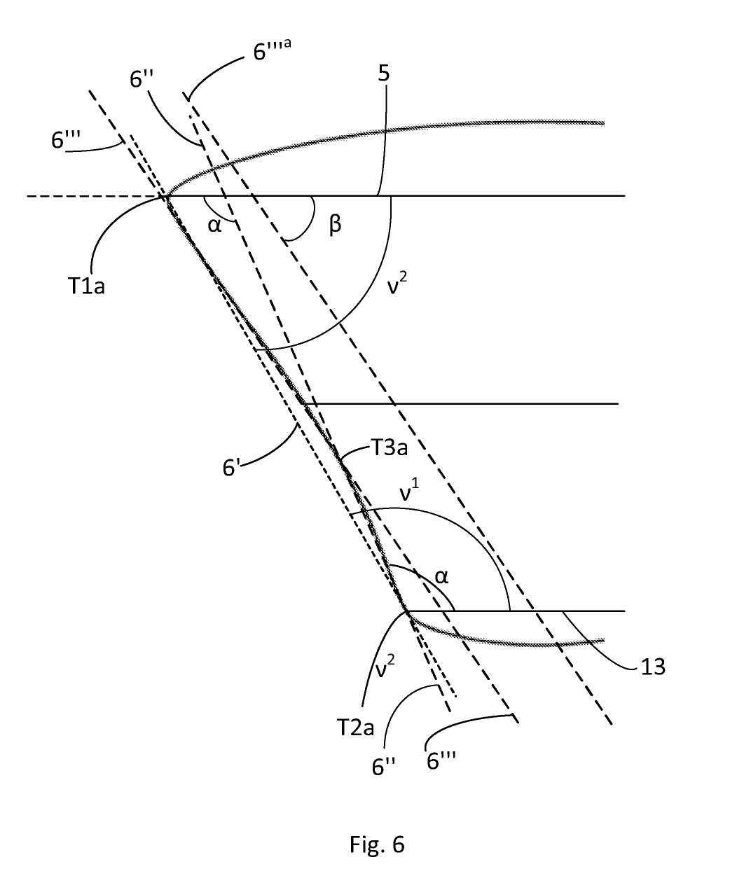

In order to further improve the fit of the veil in accordance to the wearer's anatomy, the first and second side edges 6a, 6b of the veil may advantageously have a curved concave shape between the front and rear transition points T1a, T1b, T2a, T2b. The examples of veils shown in FIGS. 4-6 have such curved side edges. A preferred curvature is shown in more detail in FIGS. 5 and 6.

As illustrated in FIG. 5, the main portion 7 of the intimate skin conditioner veil preferably comprises a front main portion 7a located between the front transversal line 5 and a straight intermediate transversal line 16, and a rear main portion 7b located between the rear transversal line 13 and the intermediate transversal line 16. The intermediate transversal line 16 is located at the longitudinal centre of the main portion, such that the longitudinal length of the front main portion 7a is the same as the longitudinal length of the rear main portion 7b. The curved concave shapes of the first and second side edges 6a, 6b preferably change curvature at respective intermediate transition points to adopt respective directions of curvature and continue to run in the same respective directions of curvature. For example, FIG. 5 shows that the curved concave shape of the first side edge 6a changes curvature at an intermediate transition point T3a, and continues to run in the same direction of curvature, and the intermediate transition point T3a is located in the rear main portion 7b between the intermediate transversal line 16 and the rear transversal line 13. The second side edge 6b has a similar shape. The main portion preferably has an intermediate transversal width W3 of 65-90 mm at the intermediate transversal line 16, in order to give a good fit to the wearer's anatomy.

The fit to the anatomy of the wearer can be further configured to adapt the curvature of the side edges of the veil to the groin anatomy of the wearer. As illustrated in FIGS. 5 and 6, the first and second side edges 6a, 6b may thus advantageously have a curvature, which changes such that a straight line 6″ between the first rear transition point T2a and the intermediate transition point T3a, is inclined in relation to the rear transversal line 13 at an angle α, which is >90°, and a straight line 6′″ between the first front transition point T1a and the intermediate transition point T3a is inclined in relation to the front transversal line 5 at angle β, which is <180°−α. Further, the angle α is preferably smaller than the angle v¹ between the rear transversal line 13 and the straight line 6′ between the first rear transition point T2a and the first front transition point (T1a), and the angle β is preferably smaller than the angle v² between the straight line 6′ and the front transversal line 5. The angle α is preferably 95-160°. A line 6′″^{a}, which is parallel to the line 6′″, is drawn in FIG. 6 for illustration purposes.

### Materials

In order to conform well to the body of the wearer and to add to the wearing comfort, the intimate skin conditioner veil 1 is preferably very pliable, thin and breathable. The veil comprises a garment facing layer arranged on the garment facing side of the veil, and a space creating layer arranged on the skin contacting side of the veil. The layers comprised in the veil are suitably connected by adhesive or ultrasound welded joints. The intimate skin conditioner veil may include fastening means positioned on the garment facing side, preferably in the form of one or more adhesive or friction areas.

The pliability, stiffness of the intimate skin conditioner veil 1 can be expressed as Gurley Units, and the veil 1 preferably has a Gurley Units of 5-300 mgf, more preferably 5-100 mgf. Gurley Units are measured according to the standard method EDANA/INDA NWSP 090.2.RO (15) modified for measuring the stiffness of an intimate skin conditioner veil product, as described in more detail below. The desired pliability can preferably be obtained by choosing the layers of the veil as described below.

The intimate skin conditioner veil 1 preferably has a total thickness of 0.6-3.8 mm. The space creating layer of the veil may be a combined layer, comprising a loft layer and a top sheet. The loft layer of the space creating layer preferably has a basis weight of 15-100 gsm and a thickness of 0.5-3.5 mm.

The top sheet is preferably a nonwoven material comprising natural and/or synthetic fibres, and preferably has a basis weight of 8-100 gsm, which may be perforated or embossed. Laminates consisting of two or more top sheet material may also be employed, and the top sheet material may be different in different parts of the skin-contacting surface.

The loft layer preferably comprises hydrophobic or hydrophilic nonwoven material, apertured thermoplastic film or open foam material. The nonwoven material may be for example, but is not limited to, air through bonded nonwoven, spunbond, SMS material (spunbond/meltblown/spunbond), carded thermobonded nonwoven, or spunlaced (hydroentangled) material.

Alternatively, the space creating layer may be a multilayer layer comprising a loft portion and a top sheet portion, the loft portion being positioned between the garment facing layer and the top sheet portion. The space creating layer may then be a nonwoven material comprising at least two layers integrated into each other, wherein the loft portion preferably comprises spunlaid filaments having a thickness greater than 2.5 dtex, giving an open structure to the loft portion, and the top sheet portion preferably comprises staple fibres, giving a soft feeling. A suitable multilayer layer is described in WO2008147264A1, the disclosure of which is incorporated herein by reference for its description of a multilayer space creating layer. Such a multilayer space creating layer preferably has a basis weight of 15-100 gsm and a thickness of 0.5-3.5 mm.

The loft layer or loft portion of the space creating layer provides an open structure, which contributes to forming a distance between the skin and the garments of the wearer. The loft layer or loft portion can thus function as a replacement for the hair that has been removed, and can thus contribute to airiness and favourable skin conditions in the genital area. All layers included in the veil can have the same size, or the loft may be smaller than the garment facing layer and the top sheet. With a smaller loft layer, the veil comprises an edge portion along the outer contour, comprising only the garment facing layer and the top sheet, thus making the edges of the veil even thinner and softer.

The garment facing layer is suitably breathable, and preferably comprises a breathable plastic film or a nonwoven material, or a laminate including a breathable plastic film and a layer of nonwoven material. The garment facing layer is preferably also liquid-tight, which means that the material can resist the flow of liquid. Suitable materials for the garment facing layer include, but are not limited to, perforated films, microporous films, macroporous films, nanoporous films, or nonwoven or laminates thereof. Examples of nonwoven laminates include, but are not limited to, laminates of spunbond and meltblown, for example SMMS laminate (spunbond/meltblown/spunbond-laminate). The garment facing layer material can include, but is not limited to, renewable material in the form of PLA starch or the like.

The intimate skin conditioner veil 1 preferably does not affect the humidity conditions that prevail in the intermediate space between the skin and the veil more than normal cotton briefs. Each layer of the intimate skin conditioner veil 1 advantageously has a moisture vapour transmission rate greater than 1000 g/m²/24 h, and the intimate skin conditioner veil preferably has a total moisture vapour transmission rate greater than 1000 g/m²/24 h, preferably 1000-6000 g/m²/24 h, more preferably 1300-1600 g/m²/24 h. The garment facing layer thereby allows perspiration generated by the skin to be able to leave the surface of the skin, which reduces moisture on the skin surface and thereby reduces the risk for undesirable skin conditions. The garment facing layer suitably has a basis weight of 18-21 gsm.

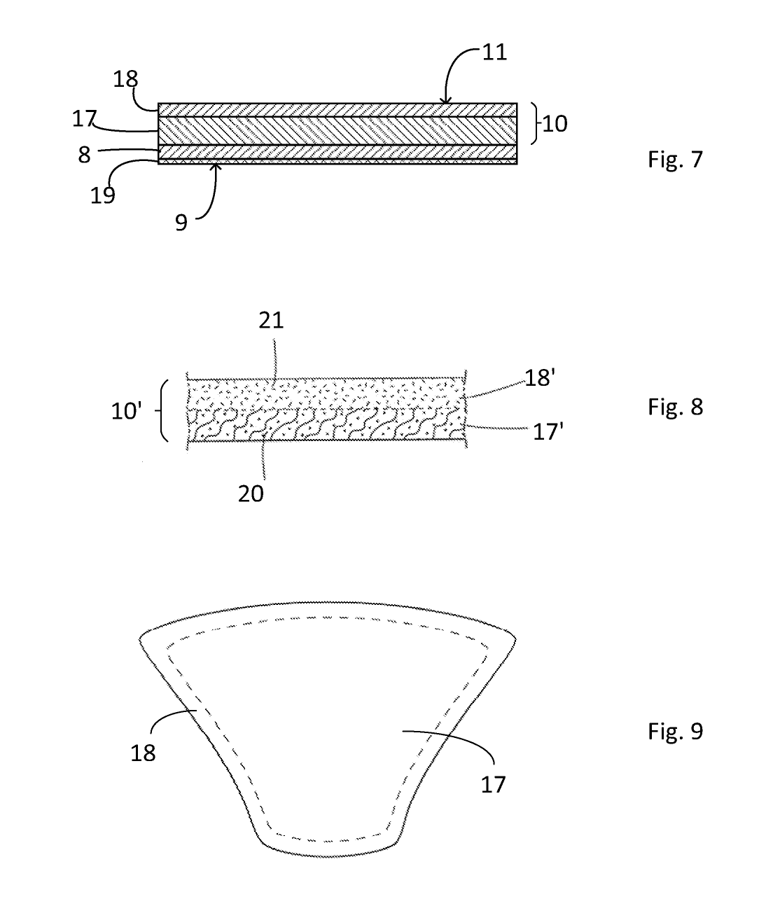

FIG. 7 shows a schematic cross sectional view of the veil. The veil 1 has a garment facing side 9 and a skin contacting side 11, the garment facing side 9 and the skin contacting side 11 facing away from one another. The veil 1 comprises a garment facing layer in the form of a back sheet 8 arranged on the garment facing side 9 of the veil, and a space creating layer 10, arranged on the skin contacting side 11 of the veil. Fastening means 19 are arranged on the garment facing side 9 of the veil. In the example shown in FIG. 7, the space creating layer is a combined layer 10, comprising a loft layer 17 and a top sheet 18, and the loft layer 17 is positioned between the garment facing layer 8 and the top sheet 18. FIG. 8 illustrates an multilayer space creating layer 10′ that can be used instead of the combined space creating layer 10 shown in FIG. 7. The multilayer space creating layer 10′ comprises a loft portion 17′ comprising spunlaid filaments 20 and a top sheet portion 18′ comprising staple fibres 21. The multilayer space creating layer 10′ is arranged in the veil such that the loft portion 17′ is positioned between the garment facing layer 8 and the top sheet portion 18′. FIG. 9 is a schematic top view, showing an example of an intimate skin conditioner veil in which the loft layer 17 is smaller than the top sheet 18 and the back sheet (not shown in FIG. 9).

### Measuring Methods

### Stiffness

All values for thickness given above for the layers comprised in the intimate skin conditioner veil are obtained by measuring by means of a circular measuring foot having a diameter of 31.5 mm at pressure of 0.2 kPa.

Gurley stiffness is measured according to the standard method EDANA/INDA NWSP 090.2.RO (15). This method is originally intended for measuring stiffness of nonwoven materials, and is therefore modified for measuring stiffness of the entire intimate skin conditioner veil. The method accordingly includes the following additional steps:
remove any release paper from the veil, and cover the attachment adhesive lightly with talcum, or the other equivalent powder;
cut a representative sample from the center region of the veil, avoiding any folds; and
if the stiffness of the veil is different along the longitudinal and the transverse axel, let the lowest value represent the article.

### Moisture Vapour Transmission Rate

The Moisture Vapour Transmission Rate is a measure of the amount of moisture adsorbed by Calcium-Chloride in a “cup” like container covered with the test specimen from controlled outside air conditions (40±3° C./75±3% relative humidity). The sample holding a cup is a cylinder with an inner diameter of 30 mm and an inside height from bottom to top flange of 49 mm. A flange having a circular opening to match the opening of the cylinder can be fixed by screws, and a silicone rubber sealing ring, matching the inner diameter, fits between the top flange and the cylinder. The test specimen is to be positioned such that it covers the cylinder opening, and can be tightly fixed between the silicone rubber sealing and the upper flange of the cylinder. The test specimen is positioned on the cylinder with the garment facing layer facing the Calcium-Chloride. When testing an intimate skin conditioner veil having adhesive areas applied to the garment facing layer, a plurality (at least 5) test specimens are cut from the veil and tested, and a value representing the MVTR for the entire product is obtained by calculating an average MVTR value of the individual test specimens. The equipment as well as the test specimen should be well adjusted to the temperatures, and the constant temperature/humidity chamber preferably has a size to accommodate up to 30 samples. The absorbent desiccant material is CaCl₂, such as can be purchased from Wako Pure Chemical Industries Ltd., Richmond, Va., US under the product designation 030-00525. If kept in a sealed bottle, it can be used directly. It also can be sieved to remove lumps, or excessive amounts of fines, if existing. It also can be dried at 200° C. for about 4 hrs. 15.0±0.02 g of CaCl₂ are weighed into the cup, and tapped lightly so as to level it out, such that the surface is about 1 cm from the top of the cup.

The samples, which are cut to about 3.2 cm by 6.25 cm, are placed flat and overlapping with the seal over the opening, and the seal and the top flange are affixed by the screws without over tightening. The total weight of the cup assembly is accurately recorded on a four decimal places scale, and the assembly is placed into the constant temperature/humidity chamber. After 5 hrs (without opening of the chamber), the sample is removed and immediately covered tightly with non-vapour permeable plastic film such as SARAN wrap as commonly used in the U.S. After about 30 mins to allow for temperature equilibration, the plastic film cover is removed and the accurate weight of the assembly is recorded. The MVTR value is then calculated from the moisture increase during these 5 hours through the 3 cm circular opening and then converted to units of g/m²/24 h. For each test, three replicates should be run, the resulting values will be averaged, and the result rounded to the nearest 100 value.

Overall, this method is applicable to thin films, multi-layer laminates and the like. Experience has shown that typical standard deviations range between 50 and 250 g/m²/24 h for averaged values of up to about 5000 g/m²/24 h. Due to this range, materials being considered to be essentially vapour impermeable, such as conventional PE films, are reported as having a MVTR of about 200 g/m²/24 h. If the units for an MVTR value are omitted for simplicity, a material “having a MVTR value of 1000” should accurately be a material “having a MVTR value of 1000 g/m²/24 h” according to this method.

Hygiene products include wipes for cleaning purposes of the skin, such as for example wet and dry wipes. Hygiene products also refer to “absorbent products,” namely products that absorb or that are adapted to absorb bodily fluids, such as urine or blood. An absorbent product may be wearable by a user. A “wearable absorbent product” refers to an absorbent article which is to be worn by the user, such as a diaper, pant-type diaper, sanitary napkin, panty-liner or incontinence product.

An “absorbent fibre” refers to a fibre having the ability to absorb liquid, such as about 1 g liquid/1 g fibres. The absorbent fibre also has moisture buffering capacity and is defined as hygroscopic.

A “non-absorbent fibre” refers to a fibre with substantially no absorption capacity.

The nonwoven material layers or webs of the present disclosure may advantageously be, for example, spunbond, air laid, wet laid, carded, electro spun or meltblown nonwovens. The nonwoven material may be bonded by multiple techniques, such as, but not limited to, by needling, hydroentangling, or heat bonding.

The nonwoven material of the disclosed products may be a mixture of natural and synthetic materials or may be comprised of only synthetic or natural fibres. Natural fibres are for instance cellulosic fibres or fibres from regenerated cellulose. Synthetic fibres are for instance polyester fibres, polyolefin-based fibres such as, but not limited to, polypropylene fibres or polyethylene fibres, and/or combinations thereof or the like.

The nonwoven material may be a combination of several types of nonwoven materials, such as, but not limited to, spunbond-meltblown, spunbond-meltblown-spunbond (SMS) type or spunbond-meltblown-meltblown-spunbond (SMMS) type. In case several layers of nonwoven materials are laminated by gluing or by ultrasound, the present description mainly relates to the nonwoven layer lying against the skin.

The basis weight for the nonwoven layer can be varied from 8 to 80 g/m², preferably from 8 to 30 g/m², and more preferably from 8 to 20 g/m². When the basis weight is under 40 g/m², sufficient breathability, drapeability and comfort for the product can be obtained. A basis weight from 8 to 20 g/m² has been found to provide best comfort and flexibility while processability of the material is still good.

The terms “lubricating” or “lubricant” refer to a substance or composition that serves to lubricate, thus making a surface onto which the composition is applied slippery.

The lubricating coating composition may reduce the wet friction.

The lubricating coating composition of the present disclosure may include silicone oil, i.e. polymerized siloxane, and preferably polydimethylsiloxane which is also referred to as dimethicone.

The lubricating coating composition may also include pentandiol, such as for example 1,5 pentandiol.

The lubricating coating composition may also include allantoin (e.g., 5-ureidohydantoin or glyoxyldiureide).

The lubricating coating composition may also include a polysaccharide such as for example beta glucan or pectin. Pectin is a polysaccharide having a mean molecular weight of 20 000-400 000 u. The polysaccharide is mainly composed of galacturonic acid units, which can be esterified to varying degrees. Pectin is naturally present in the cell walls of all plants and functions as a binding agent. A common source for extracting pectin is citrus peel.

Another suitable lubricating coating is SKINMIMICS®, which comprises ceteareth-25, glycerin, cetyl alcohol, behenic acid, cholesterol, ceramide NP, ceramide NS, ceramide, EOS, ceramide EOP, ceramide AP, caprooyl, phytosphingosine, caprooyl and sphingosine.

Furthermore, another suitable lubricating coating is SK-INFLUX® V, which comprises ceramide NP, ceramide AP, ceramide EOP, phytosphingosine, cholesterol, sodium lauroyl, lactylate, carbomer and xanthan Gum.

When fibres are coated with a lubricating coating composition at least on a surface arranged to be in contact with skin of a user during use of the absorbent product, the lubricating coating composition decreases the wettability of the fibres, and the capillarity force is decreased, resulting in that the interaction has been weakened and that a lower force is needed to break each meniscus. The lower force needed to break the menisci leads to a reduced wet friction.

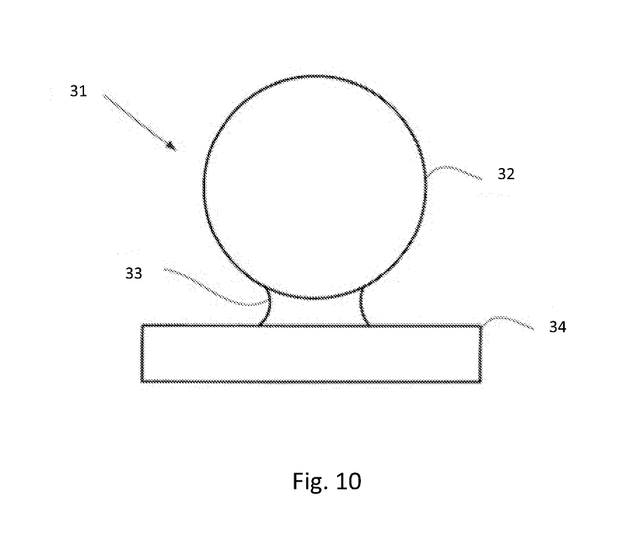

FIG. 10 schematically shows an enlargement of an illustrative embodiment of one fibre 32 in a nonwoven material 31 in the presence of moisture. FIG. 10 thus shows the effect of the presence of moisture on the fibres in a nonwoven when the nonwoven is moist. The moisture causes menisci 33 to form between the fibre 32 of the nonwoven material 31 and the skin 34 of a user, thus increasing the wet friction between the nonwoven material 31 and the skin 34. The menisci 33 form a strong interaction between the nonwoven material 31 and skin 34, i.e. a relatively strong force is needed to break each meniscus 33. This is a result of the capillarity force of the liquid around the surface contact site, which can have an important effect on the strength of adhesion joints and which can form meniscus 33. The surface contact site refers to the area of the fibres 32 contacting the liquid and the area of the skin 34 contacting the liquid

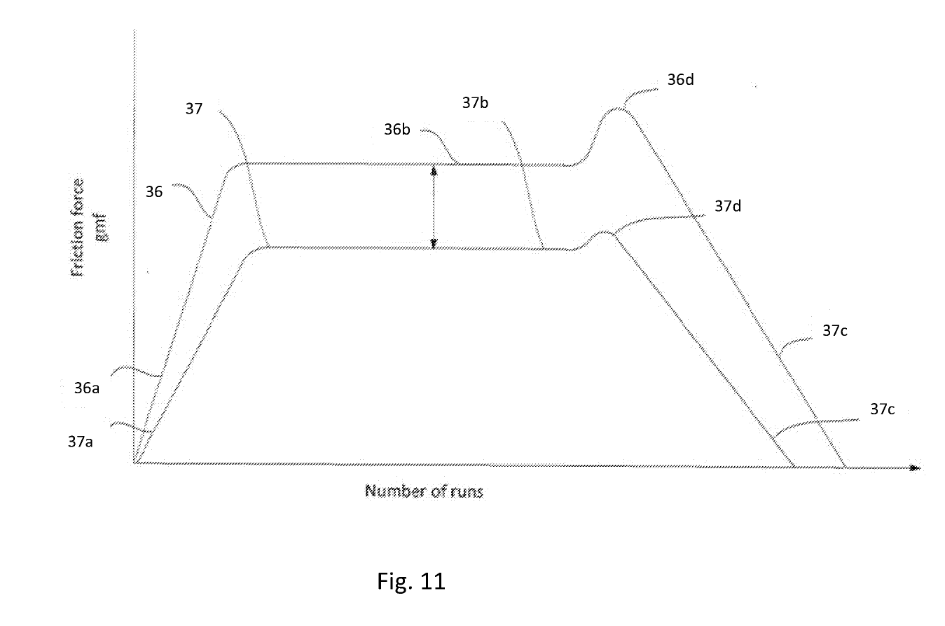

FIG. 11 schematically shows friction measurements made with an illustrative embodiment of a nonwoven material with and without the lubricating coating composition using the repeated stick and slip method. FIG. 11 shows two curves. The first curve 36 illustrates the friction values of a nonwoven material comprising only non-absorbent fibres. The second curve 37 illustrates the friction values of a nonwoven material comprising coated fibres. The friction values of curves 36, 37 are plotted on a graph with the number of friction running on the x-axis and the friction force in gmf on the y-axis. The unit “gmf” refers to a gram-force. One gram-force is 9.80665 mN.

Stick and slip measurement method for measuring the wet friction:

The stick and slip measurement method measures the static friction, i.e., sns value (stick and slip value) in gram force, gmf, between a material and the skin. The method includes making repeated runs using the same material strip. First the sns value for the dry state (dry material and skin) is measured followed by wet state at different liquid levels (from completely wetted material, to moist and to almost dry) until the sns value is back to the skin-material interaction level measured in the first dry run, which means that the material is dry again. The method is thus called a repeated stick and slip method or sns run dry-wet-dry.

Definition of the Method

The stick and slip value is defined as the point on the force curve (gmf) where the material starts gliding over the arm. The sns values from all single force curves are then put together in a new graph, sns values as a function of number of runs.

Illustrative Principle of the Method

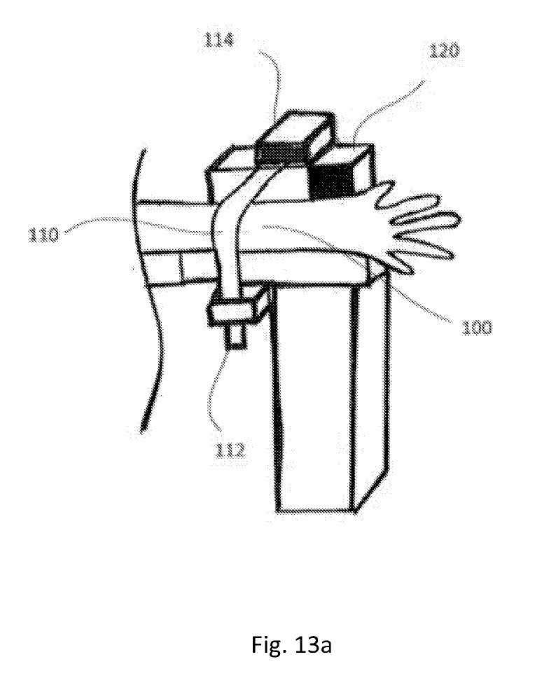

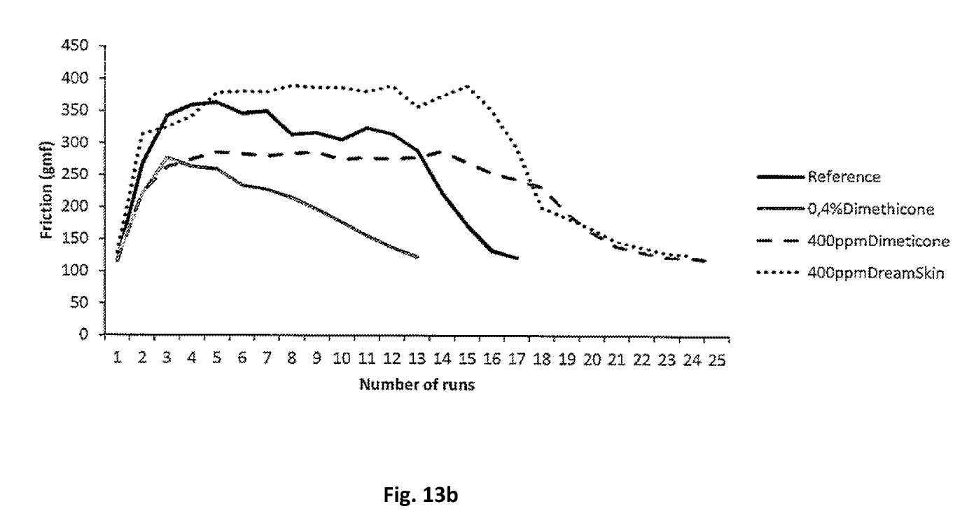

A strip 110 of test material is pulled, with the help of a MTT 170 tensile tester 120, across the volar forearm 100 to measure the static friction between the material and the skin as illustrated in FIG. 13a. First a dry strip is pulled across the volar forearm. Then the strip is wetted completely and is pulled repeatedly across the volar forearm until the dry state is reached again. Systems with dry skin/dry material, dry skin/wet material, occluded skin/dry material and occluded skin/wet material can be tested. Dry skin/wet material is the only measurement made in the present description, though during the wet runs the skin is to a certain degree influenced by the liquid in the system and may become somewhat occluded before finally reaching the level for the dry sns value again.

Illustrative Equipment of Method
Test person's arm, volar forearm
The test person is acclimatized during 15 min in a climate room with 21° C. and 45% rh.
The test is performed in a climate room with 21° C. and 45% rh
MTT 170 tensile tester from DIASTRON®
Adjustable armrest channel
Software MTT WIN® (UvWin 1.32.000)
Clamp 1: on the tensile tester
Clamp 2: counter weight, 60 g
0.9 weight % NaCl solution (150 ml/material strip)
Punch, 30*350 mm

Illustrative Material to be Tested The material to be tested is punched or made into rectangular strips measuring 30×350 mm.

When testing, the treated side, i.e. the side of the nonwoven having claimed properties, is placed towards the skin.

Wetting of the Material Strip

The material strip is completely wetted by submerging the whole strip in a beaker of 0.9 weight % NaCl solution (150 ml) for 1 min. The strip is lifted in the edge that will be placed in the clamp of the tensile tester using a pair of tweezers. An example of the clamp of the tensile tester is illustrated in FIG. 13a with reference number 114. The strip is slowly pulled up against the edge of the beaker which allows the material to drain its excess liquid. This represents a completely wetted nonwoven material, with a saturation of 100%. At the other edge of the strip the counter weight of 60 g is placed. The counter weight is illustrated in FIG. 13a with reference number 112.

The sns runs with the wet strip are then tested in the same way as the first run.

Illustrative Start Procedure

The computer and control unit are turned on and the instrument and program is initialized and is in the ready to start mode.

Delay time for every friction measurement is 12 seconds, to provide sufficient time to zero the load and place the material strip in the desired position on the arm before the run begins.

If a material strip has not reached its slip value after 50 mm, the distance needs to be increased.

Illustrative Positioning of Test Person's Arm

The test person should be standing close to the instrument with the arm supported comfortably in the armrest channel. The armrest channel is adjusted so that the top of the volar forearm is level with the clamp on the tensile tester. This means that the material strip is horizontal between the clamp and arm. During measurements, the arm should be kept still and relaxed.

Performing the Test

The dry nonwoven strip is placed in the clamp on the tensile tester, and the 60 g counter weight is fastened at the other edge of the strip.

The test person's arm should be correctly placed in the armrest channel as described above.

The test is started. The first seconds of the delay time are used to zero the measurement, lift the nonwoven strip from the arm, and hold the counter weight so that there is no tension on the tensile tester. The nonwoven strip is then hung over the relaxed arm; the counter weight should be still. The sns run starts directly when the delay time of 12 sec is finished.

The load cell travels the pre-set distance (50 mm) at a certain speed (150 mm/min) and pulls the nonwoven strip over the arm. When the load cell stops the sns value is noted. The sns value, which is where the material no longer “sticks” to the skin and starts to glide, is noted for every repetition.

The tensile tester is allowed to return to the start position.

The same nonwoven strip is then submerged in a bath of 0.9% NaCl solution for 1 min, as described above in the “Wetting of material strip” section. The wet nonwoven strip is then attached to the clamp on the tensile tester exactly in the same manner as for the first run with the counterweight at the edge and the arm in the same position. The friction measurement is started in the same way as the first run and the sns value from the friction curve for the second run is noted.

Then the nonwoven strip is lifted away from the arm, without touching anything, while the tensile tester goes back to its start position. When back at the start position the next run will be started, in the same way as for the first run and as soon as possible, and the third sns value is noted. The test continues in this manner until the sns value is on the same level as the first run.

Calculation and Expression of Results

The sns value from each run is noted (gmf) and a graph showing the repeated sns values (gmf) is made, sns values as a function of number of runs.

An illustrative embodiment of a nonwoven material having a lubricating coating composition and wherein the fibres have a coarseness of 0.1 to 10 dtex is represented in FIG. 11 by the second curve 37, and has lower relative friction values than a nonwoven material consisting of fibres without the coating having a finer coarseness than 0.1 dtex represented by first curve 36. The friction values for both materials are measured according to the repeated stick and slip method on the same test person. Curves 36, 37 have friction value measurements obtained in repeated runs using the method. The curves 36,37 comprises a first slope 36a, 37a having a positive coefficient illustrating increase in the friction values, a plateau 36b, 37b, and a second slope 36c, 37c having a negative coefficient illustrating decrease in the friction values. At the plateau 36b, 37b, the friction values are essentially constant, though small variations are possible.

The curves start at a value corresponding to the dry friction measured for dry nonwoven material. The positive coefficient of the first slope 36a, 37a indicates an increase in friction when the dry nonwoven material is wetted and wet friction occurs. The second slope 36c, 37c illustrates the interface between the skin and nonwoven returning the friction curve to the value of dry friction. FIG. 11 shows that the nonwoven material according to the present disclosure has a lower wet friction over the entire range of runs.

For some materials a very clear peak can be seen in a curve of friction values. As indicated by references 36d and 37d in FIG. 11, these peaks are caused by clinging, which occurs when only a small amount of moisture is present. The mechanical and adhesive properties of many substances are very sensitive to the presence of moist. This is the effect of the capillarity of liquid around surface contact site, which can have a profound effect on the strength of adhesion joints and which can form meniscus. This is the clinging effect and clinging force. This clinging effect may be reduced with a nonwoven material according to the present disclosure, as the clinging is mainly caused by forces acting on the material and the skin by the menisci.

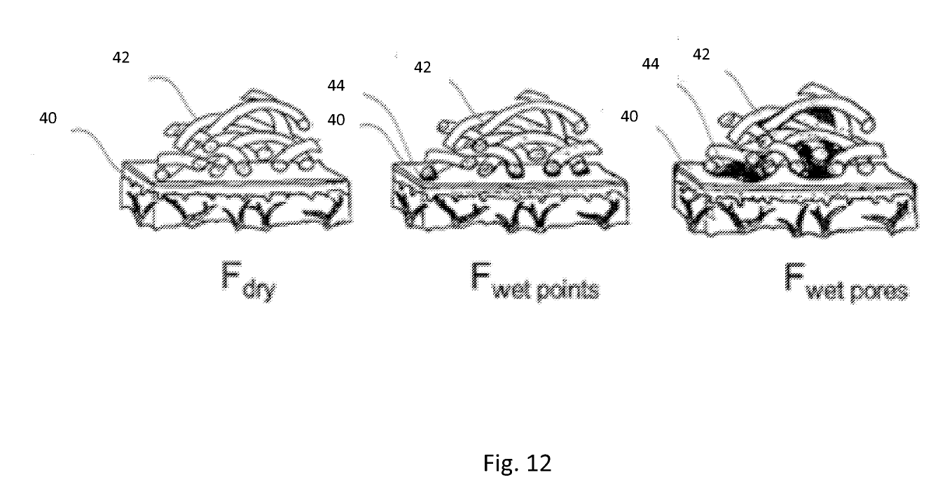

FIG. 12 shows principle sketches of the different force contributions associated with illustrative embodiments of the nonwoven material, and illustrates where on the wet-dry range these forces act. From left to right, FIG. 12 shows: F_dry (the force exerted between dry skin 40 and a dry nonwoven material comprising fibres 42), F_wet points contacts (force exerted between the fibres 42 of the material and the skin 40 in the presence of a small amount of moisture 44), and F_wet pores (the force exerted between a wet material comprising fibres 42 and a large amount of moisture 34 illustrated by the shadowed area and the skin 40). In the last case (F_wet pores) there is more moisture present than for when F_wet points contacts are exerted. The larger amount of moisture 44 present in the pores can be seen to create a thin film over the skin 40.

The total frictional force in a system involving moisture is the sum of the dry force (F_dry) and the clinging force (F_clinging):

F_friction=F_dry+F_clinging (Equation 1)

Normally F_dry<<F_clinging. The clinging force can be further divided into contributions arising from wet contacts and wet pores:

F_clinging=F_wet contacts+F_wet pores (Equation 2)

In reality, the frictional force is a mixture of all three interactions occurring in different proportions as described by Equation 3:

F_friction(s)=F_dry×C_dry+F_wet contacts×C_wet contacts(s)++F_wet pores×C_wet pores(s) (Equation 3)

where s is the degree of saturation at the interface and C is the occurrence of interactions.

When the material is wetted, the force from wet pores provides the substantial contribution to the wet friction. The wet pore force rapidly increases the friction as seen by the positive coefficient of first slope 36a, 37a of curve as shown in FIG. 11. The material slowly dries over the next runs. Over these runs the force from the wet pores still provides the greatest contribution to the friction force. This in indicated by the plateau 36b, 37b. As the material dries further, the force from the wet contacts, i.e. the menisci described above, provides a sharp rise in the friction force, as indicated by the sharp increase of friction of the peak 36d, 37d. After the peak 36d, 37d is reached, the material dries further, reducing the number of wet contacts. This quickly returns the curve to the dry friction value. This is seen by the negative coefficient of second slope 36c, 37c. If the peak is reached between two measurements, the peak may not be shown in the measurement.

FIG. 13b shows measurement data obtained by the repeated stick and slip method for a reference nonwoven material without a lubricating coating, two nonwoven materials which are the same nonwoven material as the reference, but having a lubricating coating composition dimethicone (PMX-200 Silicone fluid 1.5CS from Dow Corning Corporation) in different amounts, i.e. 400 ppm and 0.4%, respectively, and a nonwoven material (also the same nonwoven material as the reference) comprising a coating DREAMSKIN® from Intelligent Fabrics Technology, which is not lubricating. As shown in FIG. 13b, overall higher friction values are obtained for the nonwoven material without lubricating coating composition (the reference curve), whereas lower friction values are obtained for the nonwoven material with lubricating coating composition (the 0.4% Dimethicone curve and the 400 ppm Dimethicone curve). DREAMSKIN®, which is not a lubricating composition, rendered even higher friction values for the nonwoven material than the reference without any coating. This illustrates the effect of applying a lubricating coating composition to the fibres at least on a surface arranged to be in contact with the skin of a user during use of the absorbent reduces the wet friction. As described in conjuncture with FIG. 11, it can be seen that the plateau level which normally is followed by a sudden increase in friction (the “clinging peak”) before the friction force starts to drop again. Table 1 below shows measurement values for the curves depicted in FIG. 13b.

TABLE 1
Test results for nonwoven materials
with different coatings or no coating

| | | gmf | gmf | gmf |
|---|---|---|---|---|
| number | gmf | 0.4% | 400 ppm | 400 ppm |
| of run | Reference | Dimethicone | Dimeticone | DreamSkin |
| 1 | 118 | 121 | 116 | 129 |
| 2 | 267 | 220 | 223 | 314 |
| 3 | 343 | 277 | 263 | 326 |
| 4 | 359 | 264 | 275 | 343 |
| 5 | 363 | 260 | 286 | 379 |
| 6 | 347 | 234 | 284 | 381 |
| 7 | 350 | 228 | 280 | 380 |
| 8 | 314 | 215 | 284 | 390 |
| 9 | 317 | 197 | 286 | 387 |
| 10 | 306 | 177 | 275 | 387 |
| 11 | 325 | 156 | 278 | 381 |
| 12 | 316 | 138 | 276 | 390 |
| 13 | 290 | 123 | 278 | 357 |
| 14 | 223 | | 288 | 374 |
| 15 | 173 | | 271 | 390 |
| 16 | 133 | | 254 | 352 |
| 17 | 122 | | 244 | 293 |
| 18 | | | 232 | 200 |
| 19 | | | 191 | 184 |
| 20 | | | 162 | 167 |
| 21 | | | 140 | 147 |
| 22 | | | 131 | 138 |
| 23 | | | 123 | 129 |
| 24 | | | 122 | 126 |
| 25 | | | 117 | |

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

1. An intimate skin conditioner veil, configured to be positioned in an undergarment so that the veil covers a mons pubis in front of an anterior labia commissure of a female user, without covering any part of a vulval vestibule area of the user, wherein
the veil has a longitudinal central line extending in the longitudinal direction of the veil, and a first extension in the longitudinal direction, between a front end and a rear end of the veil, and a second extension in a transverse direction perpendicular to the longitudinal central line, and
wherein the veil comprises a front edge and first and second side edges intersects the first side edge at a first front transition point and intersects the second side edge at a second front transition point, the veil further comprises a main portion having a length in the longitudinal direction of the veil, the veil has a total longitudinal length, the length of the main portion being at least 50% of the total longitudinal length of the veil, wherein the main portion is bounded by a straight front transversal line between the first and second front transition points and by the first and second side edges, and tapers towards the rear end of the veil,
wherein the veil has a garment facing side and a skin contacting side, the garment facing side and the skin contacting side face away from one another, and the veil comprises a garment facing layer arranged on the garment facing side of the veil, and a space creating layer arranged on the skin contacting side of the veil, and
wherein the space creating layer comprises a nonwoven material arranged at least on the skin contacting side of the veil to be in contact with skin of the user during use of the veil, wherein the nonwoven material comprises non-absorbent fibres, wherein the non-absorbent fibres are arranged at least on the skin contacting side of the veil and have a coarseness of from 0.1 to 10 dtex, wherein the fibres and/or the nonwoven material is/are coated with a lubricating coating composition configured for use in hygiene products at least on the skin contacting side of the veil to be in contact with skin of the user during use of the veil.

2. The intimate skin conditioner veil of claim 1, wherein the fibres on the skin contacting side of the veil have a coarseness of from 0.5 to 3 dtex.

3. The intimate skin conditioner veil of claim 1, wherein the lubricating coating composition reduces the wet friction.

4. The intimate skin conditioner veil of claim 1, wherein the lubricating coating composition includes a silicone oil.

5. The intimate skin conditioner veil of claim 1, wherein the lubricating coating composition includes pentandiol.

6. The intimate skin conditioner veil of claim 1, wherein the lubricating coating composition includes allantoin.

7. The intimate skin conditioner veil of claim 1, wherein the lubricating coating composition includes a polysaccharide.

8. The intimate skin conditioner veil of claim 1, wherein the lubricating coating composition is coated in an amount of 10 ppm to 10% by weight, based on a total weight of the nonwoven material.

9. The intimate skin conditioner veil of claim 1, wherein the lubricating coating composition is applied by printing or kiss rolling to 20-100% of a total surface area of the skin contacting side of the veil.

10. The intimate skin conditioner veil of claim 1, wherein the nonwoven material is spunbond, air laid, wet laid, electro spun, carded or meltblown nonwoven.

11. The intimate skin conditioner veil of claim 1, wherein the nonwoven material has a basis weight from 8 to 80 g/m².

12. The intimate skin conditioner veil of claim 1, further comprising a rear edge which extends substantially in the transverse direction of the veil, at the rear end of the veil, the rear edge intersecting the first side edge at a first rear transition point and intersecting the second side edge at a second rear transition point, whereby the first and second side edges extend between respective ones of the front transition points and respective ones of the rear transition points, and the main portion extends in the longitudinal direction between the front transversal line and a straight rear transversal line between the first and second rear transition points, wherein the straight rear transversal line is perpendicular to the longitudinal central line.

13. The intimate skin conditioner veil of claim 1, wherein the main portion has a front transversal width and a rear transversal width, wherein the ratio W1/W2 is 1.1-6.6, and wherein the main portion has a front transversal width of 100-240 mm, and a rear transversal width of 25-95 mm.

14. The intimate skin conditioner veil of claim 1, wherein the ratio L1/L2 is 0.5-1, and wherein the longitudinal length of the main portion is 60-170 mm, and the total longitudinal length of the veil is 60-220 mm.

15. The intimate skin conditioner veil of claim 1, wherein the main portion is tapered such that a straight line between the first front transition point and the first rear transition point, is inclined in relation to the front transversal line at an angle (v²), which is smaller than 90°.

16-19. (canceled)

20. The intimate skin conditioner veil of claim 1, wherein the space creating layer is a combined layer comprising a loft layer and a top sheet, the loft layer being positioned between the garment facing layer and the top sheet, wherein the loft layer of the space creating layer has a basis weight of 15-100 gsm and a thickness of 0.5-3.5 mm.

21. The intimate skin conditioner veil of claim 1, wherein the space creating layer is a multilayer layer comprising a loft portion and a top sheet portion, the loft portion being positioned between the garment facing layer and the top sheet portion.

22. The intimate skin conditioner veil of claim 20, wherein the top sheet is a nonwoven material comprising natural and/or synthetic fibres.

23. (canceled)

24. (canceled)

25. The intimate skin conditioner veil of claim 1, wherein the loft layer is smaller than the garment facing layer and the top sheet.

26-28. (canceled)

29. The intimate skin conditioner veil of claim 4, wherein the silicone oil includes polydimethylsiloxane.

30-32. (canceled)
